(19) United States
(12) Patent Application Publication
(10) Pub. No.: US 2025/0288237 A1
(43) Pub. Date: Sep. 18, 2025

(54) PORTABLE ELECTROCARDIOGRAM DEVICE WITH TOUCHSCREEN DISPLAY

(71) Applicant: INMEDIX, INC., Normandy Park, WA (US)

(72) Inventors: Andrew James Holman, Seattle, WA (US); Steven Ross Daly, Sammamish, WA (US); Michael Glenn Loveless, Etobicoke (CA); Rade Gadzic, Etobicoke (CA); Sunghyun Lee, Toronto (CA)

(21) Appl. No.: 19/226,024

(22) Filed: Jun. 2, 2025

Related U.S. Application Data
(63) Continuation-in-part of application No. 29/960,995, filed on Sep. 3, 2024.
Application No. 29/960,995 is a continuation of application No. 29/857,903, filed on Oct. 26, 2022, now Pat. No. D1041661.

(51) Int. Cl. A61B 5/332; A61B 5/00; A61B 5/339

(52) U.S. Cl. CPC A61B 5/332; A61B 5/0006; A61B 5/339; A61B 2560/0214

(57) ABSTRACT

A portable ECG device may include a base comprising: a first portion configured to contain an ECG electronic system; and a second portion separate from the first portion and configured to contain and electrically connect ECG leads to the ECG electronic system. A portable ECG device may include a computing device enclosure configured to receive and support a computing device with a display. A portable ECG device may include a lid pivotably connected to the base and attached to the computing device enclosure, wherein the lid and the computing device enclosure are configured to enclose the second portion when the computing device is received by the computing device enclosure and the lid is in a closed position.

### CROSS REFERENCE AND INCORPORATION OF RELATED APPLICATIONS

This application is a continuation-in-part of U.S. Design application Ser. No. 29/960,995, filed on Sep. 3, 2024, which is a continuation of U.S. Design application Ser. No. 29/857,903, filed Oct. 26, 2022, issued Sep. 10, 2024 as U.S. Design Patent No. D1041661, the entire disclosures of which are hereby incorporated by reference herein for all that they contain, for all purposes. In addition, any and all applications for which a foreign or domestic priority claim is identified in the Application Data Sheet as filed with the present application are hereby incorporated by reference under 37 CFR 1.57.

### BACKGROUND OF THE INVENTION

This disclosure relates to portable medical devices, particularly portable ECG devices.

### DESCRIPTION OF THE RELATED ART

Cardiovascular diseases are among the leading causes of mortality worldwide, with early detection and continuous monitoring playing a critical role in effective treatment and management. Electrocardiograma a non-invasive diagnostic method widely used for monitoring the electrical activity of the heart. Traditional ECG systems are typically large, stationary, and require professional medical environments for operation, limiting their accessibility and real-time monitoring capabilities outside clinical settings.

With the advancement of wearable and mobile health technologies, there is a growing demand for compact, user-friendly, and portable ECG devices that enable continuous cardiac monitoring in a variety of settings. Current portable ECG solutions often suffer from limitations such as low signal fidelity, short battery life, cumbersome form factors, or lack of seamless data integration with digital health platforms. There exists a need for a portable ECG device.

### SUMMARY OF THE INVENTION

Some implementations described herein relate to a portable ECG device including: a base including: a first portion configured to contain an ECG electronic system; and a second portion separate from the first portion and configured to contain and electrically connect ECG leads to the ECG electronic system; a computing device enclosure configured to receive and support a computing device with a display; and a lid pivotably connected to the base and attached to the computing device enclosure, wherein the lid and the computing device enclosure are configured to enclose the second portion when the computing device is received by the computing device enclosure and the lid is in a closed position.

Some implementations described herein relate to a portable ECG device, wherein the computing device enclosure is configured to be removably attached to the lid.

Some implementations described herein relate to a portable ECG device, wherein the computing device enclosure is configured to removably receive the computing device.

Some implementations described herein relate to a portable ECG device, wherein the lid includes inwardly opposing rail guides configured to slidably receive the computing device enclosure.

Some implementations described herein relate to a portable ECG, wherein the computing device enclosure includes outwardly opposing rails configured to slide into and engage with the inwardly opposing rail guides of the lid.

Some implementations described herein relate to a portable ECG device, wherein the computing device enclosure includes enclosure docking magnets configured to engage with lid docking magnets of the lid and secure the computing device enclosure to the lid.

Some implementations described herein relate to a portable ECG device, wherein the lid is configured to be positioned at an angle such that the computing device enclosure can rest on the lid and the base in a display configuration.

Some implementations described herein relate to a portable ECG device, wherein the base includes a ridge, the ridge configured to contact the computing device enclosure when in the display configuration and prevent the computing device enclosure from moving from the display configuration.

Some implementations described herein relate to a portable ECG device, wherein the lid includes at least one engagement feature configured to engage with an alignment feature of the computing device enclosure thereby securing the computing device to the lid.

Some implementations described herein relate to a portable ECG device, wherein the ECG electronic system includes: a controller configured to serve as a signal acquisition unit to capture and process ECG signals from the ECG leads; and a battery configured to provide power to the processor.

Some implementations described herein relate to a portable ECG device further including a single power connector configured to supply power to both a battery of ECG electronic system and the computing device.

Some implementations described herein relate to a portable ECG device wherein the controller is configured to selectively direct power received through the single power connector to the battery or to the computing device enclosure based on a charge state of the battery and a charge state of a computing device contained within the computing device enclosure.

Some implementations described herein relate to a portable ECG device, wherein the controller is further configured to direct more power received through the single power connector to the battery than to the computing device enclosure if the battery has a lower charge state than the computing device contained within the computing device enclosure.

Some implementations described herein relate to a portable ECG device, wherein the controller is further configured to direct more power received through the single power connector to the computing device contained within the computing device enclosure than to the battery of the ECG electronic system if the computing device has a lower charge state than the battery.

Some implementations described herein relate to a portable ECG device, wherein the lid includes electrical contacts in electrical communication with the ECG electronic system, the electrical contacts configured to engage with corresponding contacts on the computing device enclosure.

Some implementations described herein relate to a portable ECG device, wherein the electronic contacts are configured to exhibit high impedance when not in contact with the corresponding contacts on the computing device.

Some implementations described herein relate to a portable ECG device, wherein the ECG electronic system is configured to be in wireless communication with a computing device.

Some implementations described herein relate to a portable ECG device further including the ECG leads connected to the ECG electronic system.

Some implementations described herein relate to a portable ECG device further including a computing device contained within the computing device enclosure.

Some implementations described herein relate to a portable ECG device, wherein the computing device is configured to display the ECG waveform calculated by a processor of the ECG electronic system.

### BRIEF DESCRIPTION OF THE DRAWINGS

The foregoing and other features of the disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings.

FIG. 1A shows a top isometric view of a portable ECG device.

FIG. 1B shows a top isometric view of the portable ECG device of FIG. 1A in a closed configuration.

FIG. 1C shows a bottom view of the portable ECG device of FIG. 1A.

FIG. 1D shows a bottom isometric view of the portable ECG device of FIG. 1A.

FIG. 1E shows a side view of the portable ECG device of FIG. 1A.

FIG. 1F shows a top isometric view of the portable ECG device of FIG. 1A with the computing device enclosure removed.

FIG. 1G shows a front isometric view of a computing device enclosure of the portable ECG device of FIG. 1A.

FIG. 1H shows a partial back isometric view of the computing device enclosure of the portable ECG device of FIG. 1A.

FIG. 1J shows a top isometric view of the portable ECG device of FIG. 1A in a display configuration.

FIG. 2 is a block diagram schematically illustrating various components of the portable ECG device of FIG. 1A.

### DETAILED DESCRIPTION OF THE PREFERRED EMBODIMENT

In the following detailed description, reference is made to the accompanying drawings, which form a part hereof. In the drawings, similar symbols typically identify similar components, unless context dictates otherwise. Thus, in some embodiments, part numbers may be used for similar components in multiple figures, or part numbers may vary from figure to figure. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein and illustrated in the Figures, can be arranged, substituted, combined, and designed in a wide variety of different configurations, all of which are explicitly contemplated and made part of this disclosure.

Reference in the specification to “one embodiment,” “an embodiment,” or “in some embodiments” means that a particular feature, structure, or characteristic described in connection with the embodiment is included in at least one embodiment of the invention. Moreover, the appearance of these or similar phrases throughout the specification do not necessarily all refer to the same embodiment, nor are separate or alternative embodiments necessarily mutually exclusive. Various features are described herein which may be exhibited by some embodiments and not by others. Similarly, various requirements are described which may be requirements for some embodiments but may not be requirements for other embodiments.

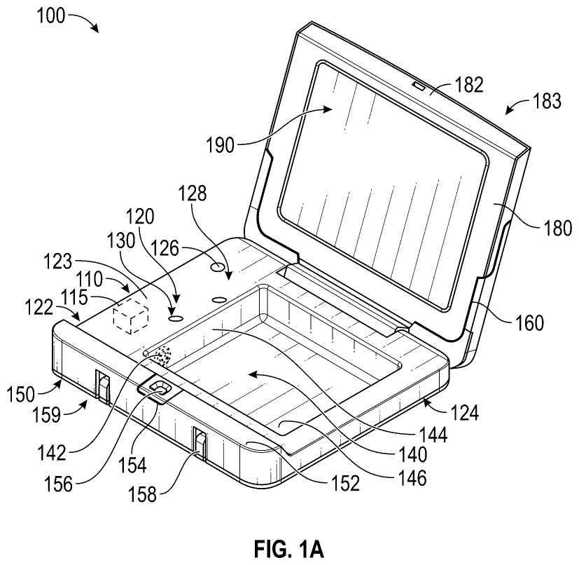

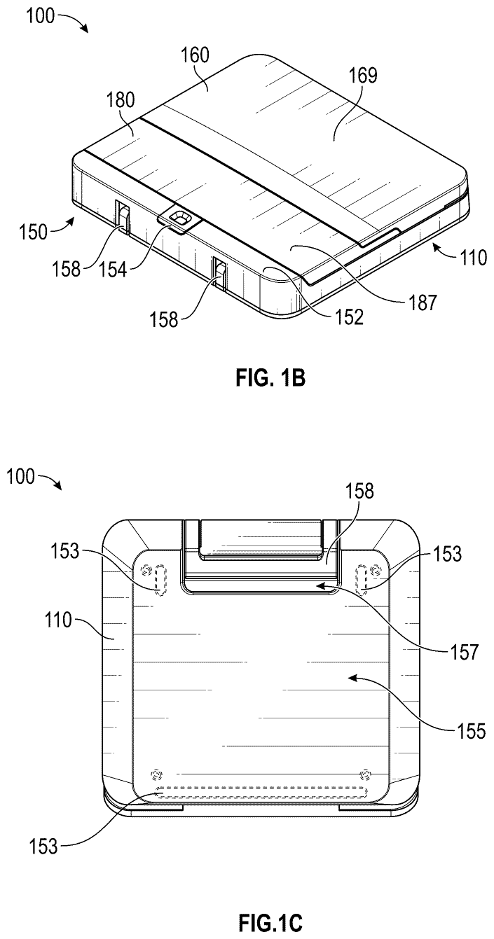

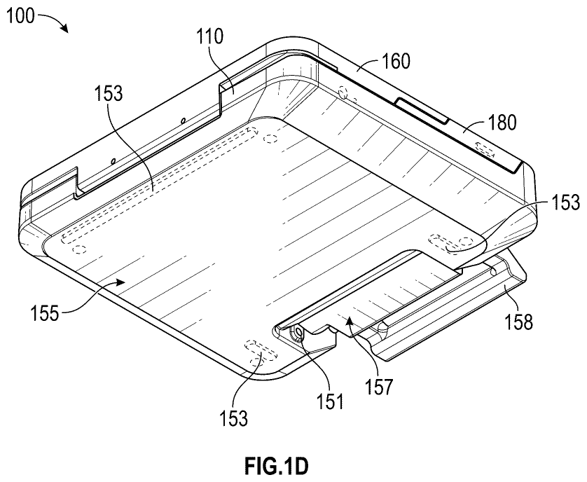

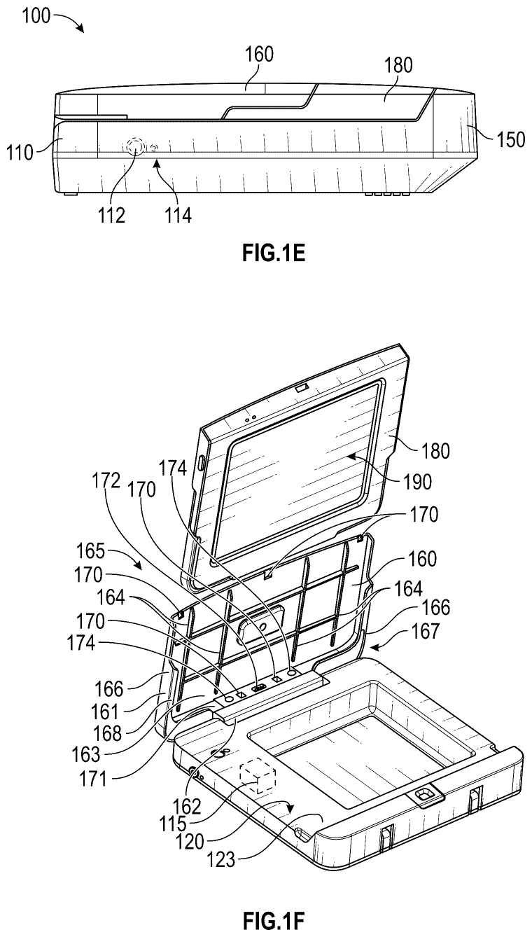

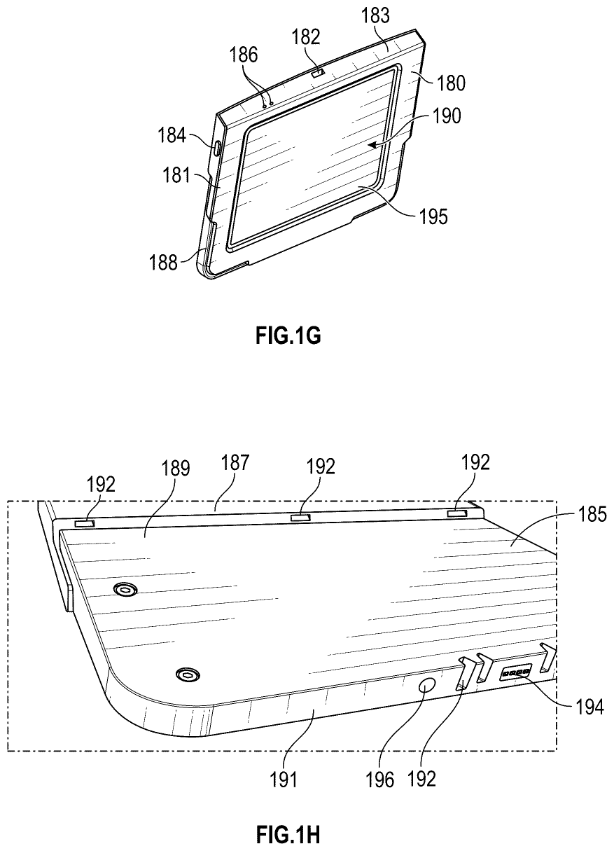

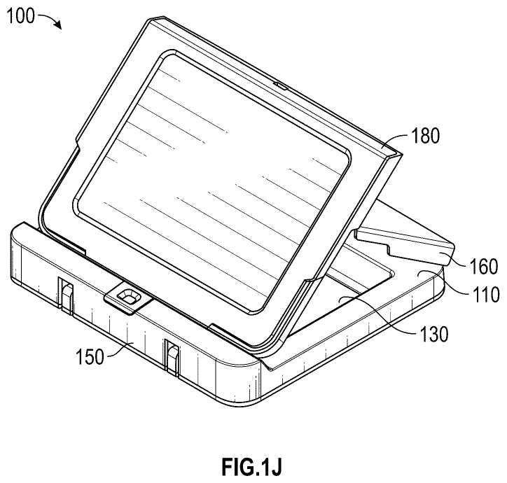

FIG. 1A shows a top isometric view of a portable ECG device 100. As seen in FIG. 1, the portable ECG device may include a base 110, a lid 160 and a computing device enclosure 180. The lid 160 and the computing device enclosure 180 are configured to engage and disengage between a docked configuration as seen in FIG. 1A where the computing device enclosure 180 is docked with the lid 160 and an undocked configuration where the computing device enclosure 180 is separated from the lid 160 as seen in FIG. 1E. When the computing device enclosure 180 is docked within the lid 160, the computing device enclosure 180 and the lid 160 form a larger cover that is configured to be rotatably connected to the base, such that the lid 160 and computing device enclosure 180 can rotate from an open position as seen in FIG. 1A and a closed position as seen in FIG. 1B. These features are described in further detail below.

FIG. 1A shows the portable ECG device 100 with the computing device enclosure 180 docked in the lid 160 and the computing device enclosure 180 and the lid 160 are in the open configuration. As seen in FIG. 1A, the base 110 may include a first portion 120 and a second portion 130. The base 110 may be generally rectangular in shape. In some embodiments, the base 110 may have another shape. The first portion 120 may be located on a first side 122 of the base 110. In some embodiments, the first portion 120 may be located on second side 124 of the base 110. In some embodiments, the first portion 120 is substantially hollow and is configured to house an ECG electronic system 115 which is described in further detail below. In some embodiments, the ECG electronic system 115 includes a battery and at least one processor. The ECG device 100 includes a power button 126 configured to selectively control power to one or more of its features. For example, the power button 126 can turn on or turn off the electronics housed within the first portion 120. In some embodiments, the power button 126 is also configured to control power to one or more other devices, such as the computing device housed within the computing device enclosure 180 when the computing device enclosure 180 is docked within the lid 160. The power button 126 can be positioned on various portions of the ECG device 100, such as on the first portion 120 as shown.

In some embodiments, the first portion 120 includes a power button LED 128. In some embodiments, the power button LED 128 is configured to be lit when the internal electronics housed within the first portion 120 are turned on. In some embodiments, the power button LED 128 is configured to be unlit when the internal electronics housed within the first portion 120 are turned off. In some embodiments, the power button LED 128 is configured to change colors based on a charge state of an internal battery (described below) contained within the first portion 120 of the base 110. In some embodiments, the power button LED 128 may be lit with a first color when the battery charge state of the internal battery is at and/or above a first threshold. In some embodiments, the first color is green. In some embodiments, the first threshold may be a 5% charge state, 10% charge state, 15% charge state, 20% charge state, 25% charge state, 30% charge state, 35% charge state, 40% charge state, 45% charge state, 50% charge state, 55% charge state, 60% charge state, 65% charge state, 70% charge state, 75% charge state, 80% charge state, 85% charge state, 90% charge state, or 95% charge state. In some embodiments, the power button LED 128 may be lit with a second color when the battery charge state of the internal battery is at and/or below a second threshold. In some embodiments, the second color may be red. In some embodiments, the second threshold may be the first threshold. In some embodiments, the second threshold may be different than the first threshold. In some embodiments, the second threshold may be a 5% charge state, 10% charge state, 15% charge state, 20% charge state, 25% charge state, 30% charge state, 35% charge state, 40% charge state, 45% charge state, 50% charge state, 55% charge state, 60% charge state, 65% charge state, 70% charge state, 75% charge state, 80% charge state, 85% charge state, 90% charge state, or 95% charge state. In some embodiments, the power button LED 128 may be lit with a third color when the battery charge state of the internal battery is at and/or below the first threshold and at and/or above the second threshold. In some embodiments, the third color is yellow.

In some embodiments, the first portion 120 includes at least one sleep magnet 130. In some embodiments, the at least one sleep magnet 130 is embedded within a surface 123 of the first portion 120 such that the at least one sleep magnet 130 is visible when the portable ECG device 100 is in the open configuration. In some embodiments, the at least one sleep magnet 130 is enclosed within the first portion 120 of the base 100 such that the at least one sleep magnet 130 is not visible on the surface 123 of the first portion 120. In some embodiments, the at least one sleep magnet 130 is configured to create a magnetic field configured to selectively cause the computing device 190 contained within the computing device enclosure 180 to enter into a sleep state (i.e., a low-power standby mode without completely shutting down the computing device 190). For example, the at least one sleep magnet 130 may cause the computing device 190 to enter a sleep state when the computing device 190 is contained within the computing device enclosure 180, the computing device enclosure 180 is docked within the lid 160, and the computing device enclosure 180 and the lid 160 are in the closed position. In some embodiments, the computing device 190 includes a Hall effect sensor. The Hall effect sensor is configured to detect the magnetic field generated by the at least one sleep magnet 130 and cause the computing device 190 to enter into the sleep state. In some embodiments, the hall effect sensor is sensitive enough to detect the magnetic field generated by the at least one sleep magnet 130 when the lid 160 and the computing device enclosure 180 are docked and are in the closed position, but not when the lid 160 and the computing device enclosure 180 are in the open position or in the display configuration as described below or when computing device enclosure 180 is separated from the lid 160.

The second portion 140 is located proximate the first portion 120. In some embodiments, the second portion is located closer to the second side 124 of the base 110 than the first side 122. In some embodiments, the second portion is located closer to the first side 122 of the base 110 than to the second side 124. In some embodiments, the second portion 140 is depressed relative to the first portion 120. In some embodiments, the second portion 140 takes the form of a depression forming a volume within the base 110. In some embodiments, the second portion 140 is in the form of a rectangle or a square, or any suitable regular or irregular shape depressed into the base 110. In some embodiments, the second portion 140 is of sufficient volume such that ECG cables can be stored within the depression without extending above the surface of the first portion 120. In some embodiments, the second portion 140 is completely enclosed and/or covered by the lid 160 and computing device enclosure 180 when in the closed configuration as seen in FIG. 1B. This facilities the containment, portability and transport of ECG cables and electrodes or other electronic or medical equipment stored within the second portion 140 of the base when the portable ECG device 100 is in the closed configuration.

In some embodiments, the second portion 140 includes at least one electronic connection 142. In some embodiments, the electronic connection 142 is disposed on side wall 144 of the second portion 140. In some embodiments, the electronic connection 142 is disposed on a bottom surface 146 of the second portion 140. The electrical connection 142 is configured to electrically connect an external component to the ECG electronic system 115 of the first portion 120 of the base 110. For example, in some embodiments, the external component is at least one ECG cable and electrode. In some embodiments, the external components are a plurality of ECG cables and electrodes. In some embodiments, a single electronic connection 142 can be configured to connect with a plurality of external components, such as a plurality of ECG electrodes. In some embodiments, the ECG cable(s) connect to the electronic connection 142 at a first end of the ECG cable and have electrode(s) disposed at the second end of the cable. The electrode(s) are configured to contact a patient and measure bio-data of the patient and send the collected data to the portable ECG device 100 via the ECG cable and the electrical connection 142. In some embodiments, the ECG cable interfaces the ECG electrode(s) with the AFE input of the ECG electronics. In some embodiments, the ECG cable is a 4-lead wire cable. In some embodiments, the cable electrode ends are color coded to indicate to which physiological location is to be attached to each electrode. In some embodiments, the ECG cable is approved for medical use and is made of biocompatible materials. In some embodiments, the ECG cable is at least 3.3 m long to allow positioning of the device on a nearby stable surface and for a cable tension free operation during measurement. In some embodiments, the ECG electrodes are placed in the patient limbs (left and right arms, left and right legs) in order to acquire patent bio-data, such as heart rate, heart rhythm and other bio-data. In some embodiments, the electrodes comprise biocompatible, disposable parts.

The base 110 can comprise a front portion 150 extending between the first side 122 and the second side 124. The front portion 150 can include a raised portion 152, which can be at a greater height relative to the surface 123 of the first portion 120. In some embodiments, the raised portion 152 extends along the entire length of the base 110 such that the entire front portion 150 is at a greater height relative to the surface 123 of the first portion 120. In some embodiments, the raised portion 152 is at a height such that when the lid 160 and the computing device enclosure 180 are in the closed position, the raised portion 150, the computing device enclosure and the lid form a substantially continuous, planar surface as seen in FIG. 1B. This provides both a pleasing aesthetic and prevents external objects from snagging on the surface.

The front portion 150 can include a connection mechanism 154. The connection mechanism 152 is configured to secure the computing device enclosure 180 to the connection portion 150 when the computing device enclosure 180 and the lid 160 are in the closed position as seen in FIG. 1B. In some embodiments, the connection mechanism 154 is a latch or a clasp. In some embodiments, the connection mechanism 154 is a hinge and spring-loaded latch. In some embodiments, the connection mechanism 154 is configured to secure the computing device enclosure 180 and the lid 160 in the closed position when the lid 160 and the computing device enclosure 180 are rotated from the open position to the closed position. In some embodiments, the connection mechanism 154 is configured to engage with a catch 182 located on the top 183 of the computing device enclosure 180, thereby securing the computing device enclosure 180 to the front portion 150 of the base 110. In some embodiments, the connection mechanism 154 includes a mechanical actuator 156. In some embodiments, the mechanical actuator 156 is configured to release the lid 160 and the computing device enclosure 180 when actuated such that the lid 160 and the computing device enclosure 180 disengage from each other, and can be rotated from the closed position to the open position. In some embodiments, the mechanical actuator 156 is actuated by a user touching or pressing the mechanical actuator 156 with a certain degree of force.

The front portion 150 can include a handle 158, for improved portability. The handle 158 can be movably (e.g., rotatably) connected to a portion of the base 110, such as the front portion 150. The handle 158 can be movably connected to the base 110 from a stored (e.g., flush) position to a usable (e.g., extended) position. In some embodiments, the handle 158 is connected to the front portion 150 near the bottom 159 of the front portion 150 and is configured to rotate from a folded configuration (as seen in FIG. 1C) to an extended configuration (as seen in FIG. 1D) as described below. In some embodiments, the handle 158 is connected to the front portion 150 by means of a friction feature, as described further herein and shown in FIG. 1D, such as a friction hinge. The friction hinge resists the rotation of the handle 158 such that the handle 158 is only rotated when a certain amount of force is applied to the handle 158. The friction hinge beneficially prevents movement and/or rotation of the handle 158 unless a certain degree of force is applied. The handle 158 beneficially provides greater portability of the portable ECG device 100 by allowing a user greater ability to transport the portable ECG device using the handle 158.

FIG. 1C shows a bottom view of the portable ECG device 100 of FIG. 1A. As seen in FIG. 1C, the handle 158 is in a stored (e.g., folded) configuration. When the handle 154 is in the folded configuration, the handle 154 is disposed within a handle aperture 157 on the bottom 155 of the portable ECG device 100. In some embodiments, the handle 158, when in the folded configuration, is disposed within the handle aperture 158 such that handle 154 is recessed relative to (e.g., does not extend beyond) the bottom surface 155 of the base 110. The handle 154 provides a pleasing aesthetic, and facilitates the placement of the portable ECG device 100 flatly on a surface without the handle 154 causing the portable ECG device 100 to be placed at an angle on the surface. This configuration can also prevents external objects from snagging on the handle 154 when the handle is in a stored position. The handle aperture 157 is a depression in the surface 153 of the bottom 155 of the base configured to receive and hold the handle 158, when the handle 158 is in the stored configuration.

As seen in FIG. 1C, the bottom 155 of the base 110 may include one or more feet 153 configured to contact a surface that the portable ECG device 100 is placed on. The one or more feet 153 beneficially allow even placement of the portable ECG device 100 on the surface. The one or more feet 153 can comprise rubber or any other suitable, stable material, for example, to prevent the ECG device 100 from sliding on an external surface during use.

FIG. 1D shows a bottom isometric view of the portable ECG 100 with the handle 158 extended. As seen in FIG. 1D, in the extended configuration, the handle 158 extends outward from the base 110 such that the handle 158 can beneficially be used to carry the portable ECG device 100. As seen in FIG. 1D, the handle aperture 157 may include at least one friction feature 151. In some embodiments, the friction feature 151 is in contact with the handle 158 when the handle 158 is in the folded configuration such that a certain amount of force is necessary to rotate the handle 158 from the stored (e.g., folded) configuration to the usable (e.g., extended) configuration.

FIG. 1E shows a side view of the portable ECG device 100 of FIG. 1A. As seen in FIG. 1E, the base 110 may include a charging port 112. The charging port 112 is configured to receive a power cord and/or be connected to a power supply and provide electrical power to the ECG electronic system 115 of the first portion 120 and/or the computing device 190. In some embodiments, the charging port 112 is configured to provide electrical power to both the ECG electronic system 115 and the computing device 190. The charging port 112 is in electrical communication with the ECG electronic system 115 of the first portion 120. In some embodiments the charging port 112 is in electrical communication with a battery housed within the first portion 120 of the base and is configured to provide electrical power to and charge the battery when the charging port 112 receives electric power from a power supply. In some embodiments, the charging port 112 is further in electrical communication with an electrical contact 172 on the lid 160, as described further herein and shown on FIG. 1F. The electrical contact 172 is configured to provide electrical power to the computing device 190 contained within the computing device enclosure 180 when docked within the lid 160. In some embodiments, the power cord received by the charging port 112 is a power adaptor configured to convert AC mains voltage into a suitable DC input for the portable ECG device 100. In some embodiments, the power adapter is rated for input voltages of 90-260 VAC±10% and input frequencies of 47-63 Hz±5%. In some embodiments, the power adapter includes a Type A or B plug. In some embodiments, the power adapter cable is about 3400 mm long. In some embodiments, the power adapter is rated for a nominal output voltage of 12 VDC with a nominal power draw of 12 W.

The base 110 may also include a charging LED 114. The charging LED 114 may be disposed proximate to the charging port 112. In some embodiments, the charging LED 128 is configured to be lit when the charging port 112 is receiving electrical power from a power supply. In some embodiments, the charging LED 114 is configured to be unlit when the charging port 112 is not connected to a power supply. In some embodiments, the charging LED 114 can be configured to change colors based on a charge state of an internal battery as described with reference to LED 128.

FIG. 1F shows the portable ECG device 100 of FIG. 1A with the computing device enclosure 180 removed from the lid 160. As seen in FIG. 1F, the lid 160 is rotatably connected to the base 110 by means of a hinge 162. In some embodiments, the hinge 162 may be a friction hinge, as described elsewhere herein with respect to the handle 158. The friction hinge resists the rotation of the lid 160 such that the lid 160 is only rotated when a certain amount of force is applied to the lid 160. The friction hinge beneficially prevents movement and/or rotation of the lid 160 unless a certain degree of force is applied. In some embodiments, the hinge 162 is configured to permit (e.g., limit) the lid 160 to rotate a certain amount. In some embodiments, the hinge 162 is configured to allow the lid 160 to rotate up to 180°. In some embodiments, the hinge 162 is configured to allow the lid 160 to rotate from a closed position to a position where the lid 160 is substantially parallel to the surface 123 of the first portion 120 and extending from the base 110. In some embodiments, the hinge 162 is configured to allowed the lid to rotate up to 120°.

The lid 160 may include one or more guiding features 164. In some embodiments, the guiding features 164 are located on an inner-facing surface of a back wall 163 of the lid 160. In some embodiments, the guiding features 164 are integrally formed with the remainder of the lid 160. The guiding features 164 may comprise thin pieces of plastic or other material that extend outwardly from the surface of the back wall 163 of the lid 160. In some embodiments, each guiding feature 164 can extend substantially between a top 165 of the lid 160 and a bottom 167 of the lid 160, and/or between the corresponding sides of the lid 160. In some embodiments, the height of each guiding feature 164 may vary along the height of the lid 160. In some embodiments, the height of a guiding feature 164 may be greater near the top 165 of the lid 160 than a height of the guiding feature 164 near the bottom 167 of the lid 160. The guiding features 164 beneficially help guide the computing device enclosure 180 when being slid into the docked position with the lid 160. The guiding features 164 are configured to contact a surface of the computing device enclosure 180 thereby facilitating sliding of the computing device enclosure 180 into the docked configuration. The guiding features 164 beneficially provide a sufficient amount of contact and number of contact points with the computing device enclosure 180, suitable to guide the computing device enclosure 180 into the docked position while simultaneously reducing the force necessary to slide the computing device enclosure 180 into the docked position, and providing stability once the computing device enclosure 180 is in the docked position.

In some embodiments, the lid 160 includes one or more rail guides, such as inwardly opposing rail guides 166. In some embodiments, a rail guide 166 is included on each corresponding opposed lateral side of the lid 160. In some embodiments, the rail guide 166 extends along a bottom portion (e.g., a portion of bottom 167), to provide additional stability. In some embodiments, the rail guides 166 are composed of three sides and are configured to partially enclose (e.g., wrap around) a portion (e.g., a portion of the opposed sides and/or bottom) of the computing device enclosure 180, thereby facilitating the sliding of the computing device enclosure 180 into the docked position. The rail guides 166 can extend substantially along the entirety of both lateral sides of the lid 160. In some embodiments, the rail guide 166 extends along a portion of the opposed lateral sides of the lid 160, and wraps around a corner bottom portion of the opposed lateral sides, and along at least a portion of bottom 167. The rail guides 166 are configured to secure the sides, and/or the bottom of the computing device enclosure 180 and to slidably receive and/or guide the sides of the computing device enclosure 180 while the computing device enclosure 180 is being slid into the docked position, and to provide stability once the enclosure 180 is in the docked position.

In some embodiments, the three sides of a portion of the rail guide 166 can be formed by a portion of the back wall 163, a side wall 161, and a lip 173. In some embodiments, the three sides of a portion of the rail guide 166 can be formed by a portion of the back wall 163, a bottom wall 171, and the lip 173. In some embodiments, the side wall 161 and or the bottom wall 171 extends at an angle (e.g., a right angle) from the back wall 163. In some embodiments, the side wall 161 extends along the length of the back wall 163 such that the side wall 161 extends from the top 165 of the lid 160 to the bottom 167 of the lid 160. In some embodiments, the bottom wall 171 extends along at least a portion of the length of the bottom 167 of the lid 160. In some embodiments, the side wall 161 can have a varied height along the length of the back wall 163. In some embodiments the height of a portion of the side wall 161 is smaller at a first location, e.g., proximate the top 165 of the lid 160 than the height of a different portion of the side wall 161 at a different location, e.g., proximate the bottom 167 of the lid 160, as shown.

The lip 168 can extend outward at an angle (e.g., a right angle) from at least a portion of the side wall 163 and/or bottom wall 171. In some embodiments, the lip 168 does not extend from the bottom to the top of the side wall. In some embodiments, the lip 168 of the rail guide 166 only extends from a portion of the side wall 161, e.g., a portion proximate the bottom 167 of the lid 160 to a point on the side wall 161 that is partway (e.g., about midway) between the bottom 167 and the top 165 of the lid 160. In some embodiments, the lip 161 extends along a first portion of the side wall 161 where the height of the side wall 161 is the greatest. In some embodiments, the lip 161 wraps around the corner of the lid 160 between the side wall 161 and the bottom wall 171 thus extending at least partially from the side wall 161 and the bottom wall 171. In some embodiments, the lip 168 does not extend along the entirety of the bottom wall 171. For example, the lip 168 can extend along the bottom wall 171 from the opposed lateral sides of the lid 160, but without a lip in a middle portion, to accommodate and provide clearance for a hinge, alignment features, electrical contact, and/or other features, as described elsewhere herein. In some embodiments, the lip 168 of the rail guide 166 only extends from a portion of the side wall 161, e.g., a portion proximate the bottom 167 of the lid 160 to a point on the side wall 161 that is partway (e.g., about midway) between the bottom 167 and the top 165 of the lid 160.

In some embodiments, the lid 160 includes one or more alignment features 170. In some embodiments, the alignment features 170 are located on the top 165 of the lid 160. In some embodiments, the alignment features 170 are protrusions that extend outwards from the top 165 of the lid 160 a certain distance. In some embodiments, the alignment features 170 are in the form of a square or a triangular protrusion. In some embodiments, a portion of the alignment features 170 can be located (in addition to or in place of the engagement features on the top 165 of the lid) on the bottom wall 171 of the lid 160. In some embodiments, the alignment features 170 are configured to engage with corresponding alignment features 192 (as seen in FIG. 1H) found on the computing device enclosure 180 thereby further securing the computing device enclosure 180 in the docked position. In some embodiments, the engagement features 170 can engage with corresponding alignment features to engage, secure, and align the computing device enclosure 180 within the lid 160 (see also FIG. 1H described below).

The lid 160 can further include an electrical contact 172. The electrical contact 172 can be located on the lid 160 such as a bottom wall 171, proximate to the hinge. In some embodiments, the electrical contact 172 comprises pogo pins. The electrical contact 172 is in electrical communication with at least a portion of the ECG electronic system 115 of the first portion 120 of the base 110 such as the internal battery and processor of the first portion 120 of the base 110. The electrical contact 172 is configured to engage with and electrically connect with a corresponding feature of the computing device enclosure 180, thereby electrically connecting the computing device enclosure 180 and the computing device 180 with the ECG electronic system 115 of the first portion 120 of the base 110. In some embodiments, the electrical contact 172 is configured to detect when the computing device enclosure 180 is docked. In some embodiments, the electrical contact 172 includes an additional feature (such as an additional pin) that is configured to detect when the computing device enclosure 180 is docked. If the electrical contact 172 determines that the computing device enclosure 180 is docked, then the electrical contact 172 allows electrical power to freely flow through the electrical contact 172 on the lid 160 to the computing device enclosure 180. However, if the electrical contact 172 determines that the computing device enclosure 180 is not docked, a series of transistors in electrical communication with the electrical contact 172 prevent a build up of voltage from accumulating on the electrical contact 172. In some embodiments, the electrical contact 172 is configured to exhibit high impedance when not in contact with the enclosure electrical contact 194. This configuration beneficially provides safety to a user by ensuring that a user cannot contact currents over 50 uA if the user accidentally comes into contact with the electrical contact 172.

In some embodiments, the lid includes one or more lid docking magnets 174. In some embodiments, the lid docking magnets 174 are located proximate to the electrical contact 172 and are configured to assist in securing the computing device enclosure 180 when docked. The lid docking magnets 174 are configured to interact with and engage with a magnetic field generated by enclosure docking magnets on the computing device enclosure 180, thereby securing the computing device enclosure 180 in the docked position. In some embodiments, the lid docking magnets 174 secure the computing device enclosure 180 to the lid 160 such that a certain amount of force is required to disengage the lid docking magnets 174 from the enclosure docking magnets on the computing device enclosure 180. In some embodiments, the force required to disengage the lid docking magnets 174 from the enclosure docking magnets can be applied by a single appendage (e.g. one hand) of a user. In some embodiments, the lid docking magnets 174 are embedded within the bottom wall 171 of the lid 160 such that the lid docketing magnets 174 are visible when the computing device enclosure 180 is not docked. In some embodiments, the lid docking magnets 174 are completely enclosed within the lid 160 below the bottom wall 171 of the lid 160 such that the lid docking magnets 174 are not visible.

FIG. 1G shows a front isometric view of a computing device enclosure 180. The computing device enclosure 180 is largely rectangular in shape and is configured to house a computing device 190. In some embodiments, the computing device 190 is an electronic display with sufficient computing power to receive and display information received from the cloud or from a portion of the portable ECG device 100. In some embodiments, the computing device 190 is a tablet such as an iPad® (as trademarked by Apple Inc.) or other suitable tablet. In some embodiments, the computing device 190 is removable disposed within the computing device enclosure 180. In some embodiments, the computing device enclosure 180 includes one or more removable or rotatable surfaces such that the computing device 190 can be accessed by a user by rotating or removing said surface.

As described further below, the computing device 190 is configured to be in wired or wireless communication with the ECG electronic system 115 contained within the base 110. In some embodiments, the computing device 190 is configured to be in further communication with a cloud server. The computing device 190 is configured to receive, process and/or display information received from the cloud server and/or the ECG electronic system 115 for convenient viewing by a user.

As described herein, the computing device 190 is contained within and removable from the computing device enclosure 180, and the computing device enclosure 180 is removable from the lid 160 and thus from the base 110 of the portable ECG device 100. This allows a user (such as a patient or a doctor) to view the computing device 190 and the data displayed thereon (such as the ECG waveform calculated from bio-data received from a patient) while the computing device 100 is separated from the rest of the portable ECG device 100. This beneficially allows for easy and convenient remote view of the data displayed on the computing device without having to move the base or other components of the ECG portable or without having to move the user. This may be especially convenient when the user is unable to move well (such as a bed-ridden patient) or to show the displayed content to persons who may be away from the portable ECG device 100 (such as to doctors or to family members of a patient in a waiting room).

The computing device enclosure 180 includes a catch 182 on the top 183 of the computing device enclosure 180. In some embodiments, the catch 182 is configured to engage with the connection mechanism 154 of the base 110 (e.g., FIG. 1A), thereby securing the computing device enclosure 180 to the front portion 150 of the base 110. In some embodiments, the catch 182 comprises a depression on the top 183 of the computing device enclosure 180 which is configured to at least partially enclose a portion of the connection mechanism 154 of the base 110, thereby securing the computing device enclosure 180 to the base 110 in the closed position.

The computing device enclosure 180 includes a power button 184. The power button 182 is configured to turn on or off the computing device 190 disposed within the computing device enclosure 180 when actuated, separately from the power button 126 for the ECG device.

In some embodiments, the computing device enclosure 180 includes at least one volume access points 186. In some embodiments, the volume access points 186 are through holes that provide access to the volume buttons of the computing device 190. In some embodiments, a user can change the volume of the computing device 190 by actuating the volume buttons of the computing device 190 by passing a pin or other similarly sized object through the volume access points 186. In some embodiments, the user can change the volume of the computing device 190 by applying pressure to the top 183 of the computing device enclosure 180 at or near the volume access points 186.

In some embodiments, the computing device enclosure includes at least one rail 188. In some embodiments, the computing device enclosure 180 includes a rail 188 on each side of the computing device enclosure 180. In some embodiments, the rails 188 are configured to engage with and slide into the rail guides 166 of the lid 160 (FIG. 1F). In some embodiments, the rails 188 are depressions in the front surface 181 and sides of the computing device enclosures that correspond to the sides of the rail guides 166. In some embodiments, when the computing device enclosure 180 is docked in the lid 160 the sides of the rail guides 166 and the front surface 181 and sides of the computing device enclosure 180 form a continuous surface as seen in FIG. 1A, for a pleasing aesthetic and to prevent snagging on external objects.

In some embodiments, the computing device enclosure 180 includes a screen 195 located centrally in the front surface 181. In some embodiments, the screen 195 allows a user to view a screen of the computing device 190 contained within the computing device enclosure 180. In some embodiments, the screen 195 may be configured to allow a user to interact with computing device 190 by touching the screen 195, effectively allowing the screen 195 to function as a touch screen. In some embodiments, the computing device enclosure 180 does not include a screen and instead the front surface 181 includes an opening in the front surface 181, thereby allowing a user to directly contact or interact with the computing device 190.

FIG. 1H shows a bottom view of the back of the computing device enclosure 180. As seen in FIG. 1H, the back of the computing device enclosure 180 includes a recessed surface 185. In some embodiments, the recessed surface 185 is recessed relative to a back surface 187 of the computing device enclosure 180. In some embodiments, the recessed surface 185 is configured to contact the guiding features 164 of the lid 160 (e.g., FIG. 1F) when the computing device enclosure 180 is being slid into the docked position. In some embodiments, the height of the recessed surface 185 is similar or equal to the height of the lid 160, such that when the computing device enclosure 180 is in the docked position, the top 165 of the lid 160 contacts the bottom 189 of the back surface 187. In some embodiments, the back surface 169 of the lid 160 (not seen) (see in FIG. 1B) forms a substantially continuous surface with the back surface 187 of the computing device enclosure 180 when the computing device enclosure 180 is docked, for a pleasing aesthetic and to prevent snagging on external objects.

In some embodiments, the computing device enclosure 180 includes one or more alignment features 192 located on a bottom 189 of the back surface 187. In some embodiments, a least a portion of the one more alignment features 192 (in addition to the alignment features 192 located on a bottom 189 of the back surface 187 or in place of) may be located on the bottom surface 191 of the computing device enclosure. In some embodiments, the alignment features 192 are depressions formed in the surface of the bottom 189 and/or in the bottom surface 191 and are configured to receive the engagement features 170 of the lid 160 (FIG. 1F) when the computing device enclosure 180 is docked, thereby helping to secure the computing device enclosure 180 in the docked position.

In some embodiments, the computing device enclosure 180 includes an enclosure electrical contact 194. The enclosure electrical contact 194 is located on a bottom surface 191 of the computing device enclosure 180. The enclosure electrical contact 194 is in electrical communication the computing device 190 and is configured to transmit electrical power and signals to and/or from the computing device 190. The enclosure electrical contact 194 is configured to engage with and electrically connect with the electrical contact 172 of the lid 160, thereby electrically connecting the computing device enclosure 180 and the computing device 190 with the ECG electronic system 115 of the first portion 120 of the base 110, such as the internal battery and the processor.

In some embodiments, the computing device enclosure 180 includes one or more enclosure docking magnets 196. In some embodiments, the enclosure docking magnets 196 are located proximate to the enclosure electrical contact 194 and are configured to assist in securing the computing device enclosure 180 when docked. The enclosure docking magnets 196 are configured to interact with and engage with a magnetic field generated by the lid docking magnets 174 on the lid 160, thereby securing the computing device enclosure 180 in the docked position. In some embodiments, the enclosure docking magnets 196 secure the computing device enclosure 180 to the lid 160 such that a certain amount of force is required to unsecure the enclosure docking magnets 196 from the lid docking magnets 174 on the lid 160. In some embodiments, the force required to unsecure the lid docking magnets 174 from the enclosure docking magnets 196 can be applied by a single appendage (e.g. one hand) of a user. In some embodiments, the enclosure docking magnets 196 are embedded within the bottom surface 191 of the computing device enclosure 180 such that the enclosure docketing magnets 196 are visible when the computing device enclosure 180 is not docked. In some embodiments, the enclosure docking magnets 196 are completely enclosed within the computing device enclosure 180 below the bottom surface 191 of the computing device enclosure 180 such that the enclosure docking magnets 196 are not visible. [0071] FIG. 1J shows the portable ECG device 100 in a display configuration. As seen in FIG. 1J, when in the display configuration, the computing device enclosure 180 is undocked and placed at an angle such that the bottom surface 191 contacts the raised portion 152 of the front portion 150 of the base 110. The lid 160 is rotated to a first position in between the closed configuration and the open configuration. In some embodiments, the lid 160 is rotated up to 30°, up to 45°, up to 60° or up to 90° from the closed position to the display configuration. In some embodiments, the engagement features 170 of the lid 160 contact the bottom 189 of the back surface 189 of the computing device enclosure 180 thereby allowing the computing device enclosure 180 to rest in the angled position. This configuration beneficially allows a user to display the computing device enclosure 180 at an angle that permits easier viewing of the computing device 190 contained in the computing device enclosure 180.

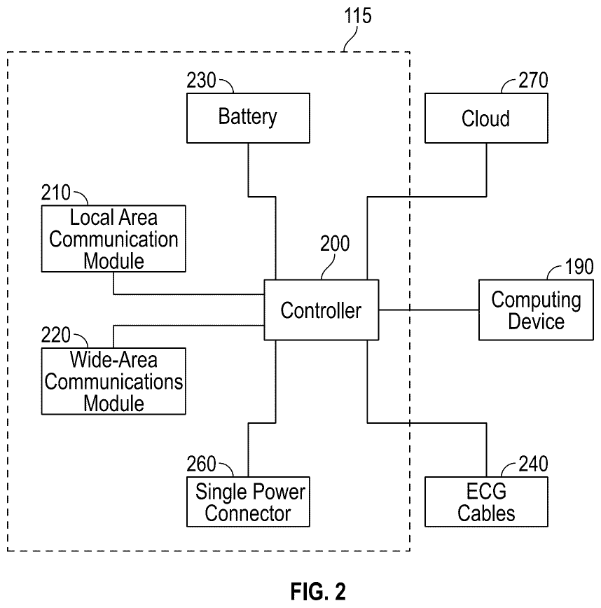

FIG. 2 is a block diagram schematically illustrating various components of the portable ECG device 100 of FIG. 1A-FIG. 1J including components of the ECG electronic system 115. The ECG electronic system 115 is represented by the dashed box in FIG. 2 and the components of the ECG electronic system 115 are portrayed within the dashed box. The ECG electronic system 115 includes a controller 200 which can be used in autonomously or manual controlling the operation of the portable ECG device 100. The controller 200 may include one or more processors, integrated circuits, field-programmable gate array or any other suitable control circuitry. In the illustrated embodiment, the controller 200 includes or is in communication with a local area communications module 210 and a wide area communications module 220. The local area communications module 210 may include one or more transceivers able to communicate via Bluetooth, Wi-Fi, or any other suitable local area communication protocol with local devices, such as the computing device 190 when not docked in the lid 160 as described in greater detail elsewhere herein. The wide-area communications module 220 may include one or more transceivers able to communicate via 4G, 5G, Edge or any other suitable wide area communication protocol with external devices, including remote devices such as cloud servers or other network entities.

As seen in FIG. 2, the controller 200 can be in wireless or wired communication with the battery 230. In some embodiments, the ECG electronic system 115 includes the battery 230. The battery 230 is disposed within the first portion 120 of the base 110 as described herein and is configured to provide electrical power to the various components of the portable ECG device 100, such as the controller 200. In some embodiments, the battery is configured to send a signal to the controller 200 to provide information to the controller 200. In some embodiments, the information includes the charge state of the battery 230 and/or whether the battery 230 is being charged actively by an external power source via the charging port 112 described above. In some embodiments, the battery 230 is configured to send the signal to the controller in response to a signal received from the controller 200. In some embodiments, the battery 230 is configured to send a signal to the controller 200 periodically. In some embodiments, the battery 230 is in electrical communication with the computing device 250 when the computing device 250 is docked. In some embodiments, the controller 200 is configured to command the battery 230 to provide power to the computing device 250. In some embodiments, the controller 200 is configured to command the battery 230 to provide power to the computing device 250 when the charge state of the computing device drops below a certain threshold.

The controller 200 is in wired or wireless communication with one or more ECG cables when the one or more ECG are electrically connected to the portable ECG device via the electrical connection described above. In some embodiments, the controller 200 is configured to send signals to and receive signals from the ECG cables. In some embodiments, the signals include patient bio-data, such as, but not limited to, heart rate, heart rhythm, and/or electrical conduction timing, received from the ECG cables attached to a patient. In some embodiments, the controller 200 converts patient bio-signals into digital format for sending to the computing device via Bluetooth. In some embodiments, the controller 200 streams data from three differential inputs and produces standard limb vector leads. In some embodiments, the controller 200 stores the collected patient bio-data in a ring buffer before limited processing: the data are shifted according to an averaged baseline and encoded to minimize bandwidth.

In some embodiments, the controller 200 receives data from the ECG cables at a sampling frequency of 500 Hz to mitigate jitter and ensure accurate R-wave detection. In some embodiments, the controller 200 has a built-in right leg drive (RLD) feature to couple the inputs, maintaining a common mode voltage and ensuring input signals remain within the common mode input range. In some embodiments, the controller 200 uses a typical RLD topology where the common mode voltage for each of the input channels is averaged, inverted with gain applied, and returned to the patient centered around the circuit's voltage reference point. In some embodiments, the controller 200 receives the bio-data from the ECG cables and calculates an ECG waveform from the bio-data. In some embodiments, the controller 200 detects the R-waves in the bio-data and creates a simple array of RR intervals that serves as the input to the HRV index calculation algorithms. These algorithms incorporate a combination of statistical, geometric, and spectral techniques to generate indices in the frequency and time domain, which can form the basis of the ECG waveform. In some embodiments, the controller 200 then sends the ECG waveform to the computing device 190 which can then be displayed on the screen of the computing 190.

As seen in FIG. 2, the controller 200 is in wireless or wired communication with the computing device 190. In some embodiments, the controller 200 is in wired communication with the computing device 190 when the computing device 190 is docked in the lid 160 as described above. In some embodiments, the controller is in wireless communication with the computing device 190 (e.g. via Bluetooth) when the computing device 190 is separated from the lid 160. In some embodiments, the controller 200 is configured to send and receive electric signal to and/or from the computing device 190. In some embodiments, the computing device 190 is configured to send signals to the controller 200 to provide information to the controller 200. In some embodiments, the information includes the charge state of the computing device 190 and/or whether the computing device 190 is being charged actively by an external power source via the charging port 112 or via the battery 230 as described above. In some embodiments, the computing device 190 is configured to send the signal to the controller 200 in response to a signal received from the controller 200. In some embodiments, the computing device 190 is configured to send a signal to the controller 200 periodically. In some embodiments, the controller 200 is configured to send an ECG waveform calculated from bio-data received from the ECG cables 240 to the computing device 190. The computing device 190 is then configured to display the waveform on a screen of the computing device 190.

As seen in FIG. 2, the controller 200 is in wireless or wired communication with the computing device with a single power connector 260. The single power connector 260 is located within the first portion 120 of the base 110. In some embodiments, the single power connector 260 forms part of the ECG electronic system 115. The single power connector 260 is in electrical communication with the battery 230 and with the computing device 190 when the computing device 190 is docked. The single power connector 260 is in electric communication with the charging port 112 and is configured to receive the electrical power received through the charging port 112 from a power supply and direct the electrical power received to the battery 230 of the computing device 190. In some embodiments, the controller 200 is configured to command the single power connector 260 to direct power to the battery 230 and/or the computing device 190 based on a charge state of the battery 230 and/or a charge state of the computing device 190. In some embodiments, the controller 200 is configured to command the single power connector 260 to direct power to the battery 230 when the computing device 190 is not docked. In some embodiments, the controller 200 may receive the charge state of each of the computing device 190 and the battery 230 and command the single power connector to direct electrical power to the device (the computing device 190 or the battery 230) with the lower charge state. In some embodiments, the controller 200 is configured to command the single power connector 260 to continuously direct power to the battery 230 until the charge state of the computing device 190 drops below a certain threshold. In some embodiments, the controller 200 is configured to command the single power connector 260 to continuously direct power to the computing device 190 until the charge state of the battery 230 drops below a certain threshold. In some embodiments, the controller 200 is further configured to direct more power received through the single power connector 260 to the battery 230 than to the computing device 190 if the battery has a lower charge state than the computing device 190 contained within the computing device enclosure 180. In some embodiments, the controller 200 is further configured to direct more power received through the single power connector 260 to the computing device 190 contained within the computing device enclosure 180 than to the battery 230 of the ECG electronic system if the computing device 190 has a lower charge state than the battery 230.

In some embodiments, the controller 200 is in wired or wireless communication with a cloud sever 270. In some embodiments, the controller 200 is configured to send and receive electric signal to and/or from the cloud server 270. In some embodiments, the controller 200 may send a signal to the cloud server 270 containing information and/or requesting information from the cloud server 270. In some embodiments, the cloud server 270 may receive information from the controller 200 containing bio-data collected by the ECG cables. In some embodiments, the cloud server 270 may receive ECG wavefroms and/or indices as calculated by the controller 200. The cloud server 270 is configured to store the information and/or data received from the controller 200. In some embodiments, the cloud server 270 is configured to send a signal to the controller 200 in response to a signal received from the controller 200. In some embodiments, the cloud server 270 is configured to send bio-data, ECG waveforms and or indices stored on the cloud server 270 to the controller 200 upon receiving a request from the controller 200.

In some embodiments, the computing device 190 is in wired or wireless communication with the cloud server 270. In some embodiments, the computing device 190 is configured to send and receive electric signal to and/or from the cloud server 270. In some embodiments, the computing device 190 may send a signal to the cloud server 270 containing information and/or requesting information from the cloud server 270. In some embodiments, the cloud server 270 is configured to send a signal to the computing device 190 in response to a signal received from the computing device 190. In some embodiments, the cloud server 270 is configured to send bio-data, ECG waveforms and/or indices stored on the cloud server 270 to the computing device 190 upon receiving a request from the computing device 190. The computing device 190 is then configured to display the received bio-data, ECG waveforms and/or indices on a display screen of the computing device 190.

The foregoing description details certain embodiments. It will be appreciated, however, that no matter how detailed the foregoing appears in text, the development may be practiced in many ways. It should be noted that the use of particular terminology when describing certain features or aspects of the development should not be taken to imply that the terminology is being re-defined herein to be restricted to including any specific characteristics of the features or aspects of the development with which that terminology is associated.

While the above detailed description has shown, described, and pointed out novel features of the development as applied to various embodiments, it will be understood that various omissions, substitutions, and changes in the form and details of the device or process illustrated may be made by those skilled in the technology without departing from the intent of the development. The scope of the development is indicated by the appended claims rather than by the foregoing description. All changes which come within the meaning and range of equivalency of the claims are to be embraced within their scope.

What is claimed is:

1. A portable ECG device comprising:
a base comprising:
a first portion configured to contain an ECG electronic system; and
a second portion separate from the first portion and configured to contain and electrically connect ECG leads to the ECG electronic system;
a computing device enclosure configured to receive and support a computing device with a display; and
a lid pivotably connected to the base and attached to the computing device enclosure, wherein the lid and the computing device enclosure are configured to enclose the second portion when the computing device is received by the computing device enclosure and the lid is in a closed position.

2. The portable ECG device of claim 1, wherein the computing device enclosure is configured to be removably attached to the lid.

3. The portable ECG device of claim 1, wherein the computing device enclosure is configured to removably receive the computing device.

4. The portable ECG device of claim 2, wherein the lid comprises inwardly opposing rail guides configured to slidably receive the computing device enclosure.

5. The portable ECG of claim 4, wherein the computing device enclosure comprises outwardly opposing rails configured to slide into and engage with the inwardly opposing rail guides of the lid.

6. The portable ECG device of claim 1, wherein the computing device enclosure comprises enclosure docking magnets configured to engage with lid docking magnets of the lid and secure the computing device enclosure to the lid.

7. The portable ECG device of claim 1, wherein the lid is configured to be positioned at an angle such that the computing device enclosure can rest on the lid and the base in a display configuration.

8. The portable ECG device of claim 7, wherein the base comprises a ridge, the ridge configured to contact the computing device enclosure when in the display configuration and prevent the computing device enclosure from moving from the display configuration.

9. The portable ECG device of claim 1, wherein the lid comprises at least one alignment feature configured to engage with an alignment feature of the computing device enclosure thereby securing the computing device to the lid.

10. The portable ECG device of claim 1, wherein the ECG electronic system comprises:
a controller configured to serve as a signal acquisition unit to capture and process ECG signals from the ECG leads; and
a battery configured to provide power to the processor.

11. The portable ECG device of claim 1 further comprising a single power connector configured to supply power to both a battery of the ECG electronic system and the computing device.

12. The portable ECG device of claim 11 wherein the controller is configured to selectively direct power received through the single power connector to the battery or to the computing device enclosure based on a charge state of the battery and a charge state of a computing device contained within the computing device enclosure.

13. The portable ECG device of claim 12, wherein the controller is further configured to direct more power received through the single power connector to the battery than to the computing device enclosure if the battery has a lower charge state than the computing device contained within the computing device enclosure.

14. The portable ECG device of claim 12, wherein the controller is further configured to direct more power received through the single power connector to the computing device contained within the computing device enclosure than to the battery of the ECG electronic system if the computing device has a lower charge state than the battery.

15. The portable ECG device of claim 1, wherein the lid comprises electrical contacts in electrical communication with the ECG electronic system, the electrical contacts configured to engage with corresponding contacts on the computing device enclosure.

16. The portable ECG device of claim 1, wherein the electronic contacts are configured to exhibit high impedance when not in contact with the corresponding contacts on the computing device.

17. The portable ECG device of claim 1, wherein the ECG electronic system is configured to be in wireless communication with a computing device.

18. The portable ECG device of claim 1 further comprising the ECG leads connected to the ECG electronic system.

19. The portable ECG device of claim 1 further comprising a computing device contained within the computing device enclosure.

20. The portable ECG device of claim 19, wherein the computing device is configured to display the ECG waveform calculated by a processor of the ECG electronic system.

21. The portable ECG device of claim 2 further comprising a single power connector configured to supply power to both a battery of the ECG electronic system and the computing device.

22. The portable ECG device of claim 21 wherein a controller of the ECG electronic system is configured to selectively direct power received through the single power connector to the battery or to the computing device enclosure based on a charge state of the battery and a charge state of a computing device contained within the computing device enclosure.

23. The portable ECG device of claim 22, wherein the computing device is configured to wirelessly receive and display an ECG waveform calculated by a processor of the ECG electronic system.
